(19) United States
(12) Patent Application Publication
(10) Pub. No.: US 2015/0150593 A1
(43) Pub. Date: Jun. 4, 2015

(54) NESTED CANNULA DEVICE FOR LUNG COLLAPSE

(71) Applicant: KONINKLIJKE PHILIPS N.V., EINDHOVEN (NL)

(72) Inventors: Karen Irene Trovato, Putnam Valley, NY (US); Kongkuo Lu, Briarcliff Manor, NY (US)

(73) Assignee: Koninklijke Philips N.V. a corporation

(21) Appl. No.: 14/405,235

(22) PCT Filed: Jun. 14, 2013
(86) PCT No.: PCT/IB2013/054866

§ 371 Date: Dec. 3, 2014

Related U.S. Application Data
(60) Provisional application No. 61/659,607, filed on Jun. 14, 2012.

(51) Int. Cl. A61B 17/34; A61M 1/00; A61M 25/06

(52) U.S. Cl. CPC A61B 17/3415; A61M 25/0662; A61M 1/008

(57) ABSTRACT

A surgical tool set for treating a collapsed lung condition employs a trocar (20), and a nested cannula including a pleural port tube (30) and a fluid suction tube (50). In operation, the trocar (20) is nested within the pleural port tube (30), and utilized to puncture a port into a pleural cavity (10) of the patient while securing the pleural port tube (30) within the port. Subsequent to removing the trocar (20) from the pleural port tube (30), a fluid suction tube (50) is advanced with a fixed orientation through the secured pleural port tube (30) in a direction of a pleural cavity (10) target location (15) and air is suctioned from the pleural air space through one or more perforated holes into the fluid suction tube (50). The fluid suction tube (50) may have a target orientation section (42) or an additional target orientation tube (40) may be used to fix the orientation of the fluid suction tube (50) relative to the pleural port tube (30).

The present invention generally relates to nested cannula designs for patients experiencing lung collapse, typically caused by air, blood or fluid in the pleural cavity. The present invention specifically relates to a standardized set of cannula tubes employing a fluid suction tube having a fixed orientation relative to a pleural port tube to reach a target location within a pleural cavity of a patient to facilitate proper removal of air, blood or other fluids so that the lung may re-expand.

Under normal conditions, the pleural cavity surrounding the lung has a lower pressure than within the lung. The lower pressure in the pleural cavity pulls the lung surface toward the chest wall, similar to vacuum pressure. As the chest and ribs expand, the vacuum pressure in the pleural cavity increases, pulling the lung surface outward. This in turn expands the lung airways and alveoli. The reverse process occurs during exhalation. However, if any part of the pleural cavity is filled with air, blood or other fluid, then the vacuum pressure drops and the lung surface cannot be held closely to the chest wall. The result is a collapsed lung.

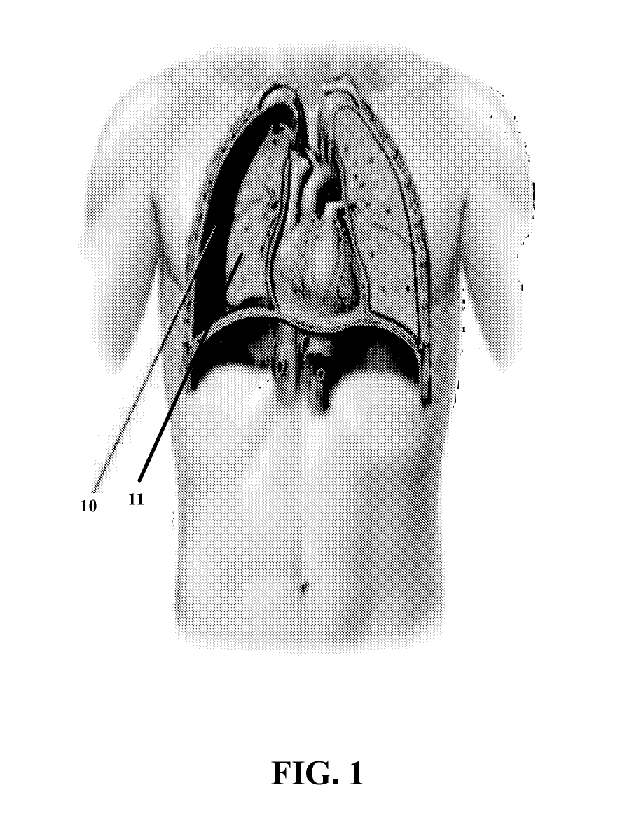

Under a collapsed lung condition as exemplarily shown in FIG. 1, air enters a pleural cavity 10 via a hole in a lung 11 or in the chest wall 12 (e.g., a gunshot hole), lowering vacuum pressure in pleural cavity 10. As a result of air, blood or other fluid entry into the pleural cavity 10, the vacuum is lost/released and lung 11 collapses.

There are many potential causes for a collapsed lung condition, such as, for example, an traumatic injury to the lung or chest wall or a lung disease such as emphysema. Irrespective of the cause of the collapsed lung condition, a hollow chest tube may be used to remove any fluid from the pleural cavity, particularly if a large area of a lung has collapsed. Typically, the chest tube is inserted between the ribs into the fluid-filled pleural cavity and a suction device attached to the chest tube removes the fluid from the chest cavity, enabling the lung to re-expand.

Many potential problems exist with the current use of a hollow chest tube, such as, for example, a puncturing of the collapsed lung during the insertion of the chest tube. However, while a customized chest tube based on image of the pleural cavity may avoid any puncture of the collapsed lung and/or an image based insertion of the chest tube may avoid any puncture of the collapse lung, a collapsed lung condition may occur suddenly and without warning and must be rapidly corrected for the patient to breathe properly. In such a case, a customized chest tube and/or image based tracking of a chest tube would be impractical, slow solutions for correcting the collapsed lung condition.

The present invention addresses a rapid correction of a collapsed lung condition (e.g., pneumothorax treatment) by providing a standardized nested cannula design that may be used immediately upon diagnosis of the collapsed lung condition.

One form of the present invention is a surgical tool set for treating a collapsed lung condition employing a trocar, and a nested cannula including a pleural port tube and a fluid suction tube. In operation, the trocar is nested within the pleural port tube, and utilized to puncture a port into the pleural cavity of the patient while securing the pleural port tube within the port. Subsequent to removing the trocar from the pleural port tube, a fluid suction tube is advanced with a fixed orientation through the secured pleural port tube in a direction of a pleural cavity target location and fluid (e.g., air and blood) is suctioned from the pleural cavity through one or more perforated holes of the fluid suction tube. The fluid suction tube may have a target orientation section or an additional target orientation tube may be used to fix the orientation of the fluid suction tube relative to the pleural port tube.

The foregoing form and other forms of the present invention as well as various features and advantages of the present invention will become further apparent from the following detailed description of various embodiments of the present invention read in conjunction with the accompanying drawings. The detailed description and drawings are merely illustrative of the present invention rather than limiting, the scope of the present invention being defined by the appended claims and equivalents thereof.

FIG. 1 illustrates a collapsed lung condition for a patient.

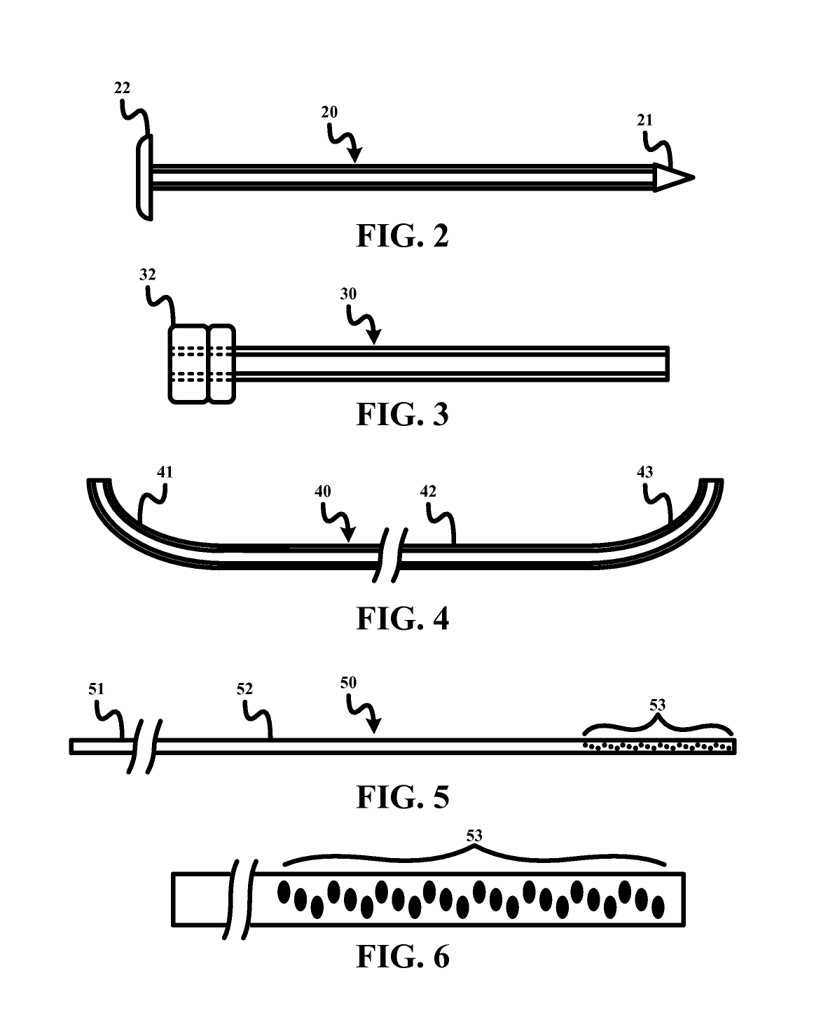

FIG. 2 illustrates an exemplary embodiment of a trocar in accordance with the present invention.

FIG. 3 illustrates an exemplary embodiment of a pleural port tube in accordance with the present invention.

FIG. 4 illustrates an exemplary embodiment of a target orientation tube in accordance with the present invention.

FIG. 5 illustrates an exemplary embodiment of a fluid suction tube in accordance with the present invention.

FIG. 6 illustrates an exemplary embodiment of a fluid suction section of the fluid suction tube shown in FIG. 5.

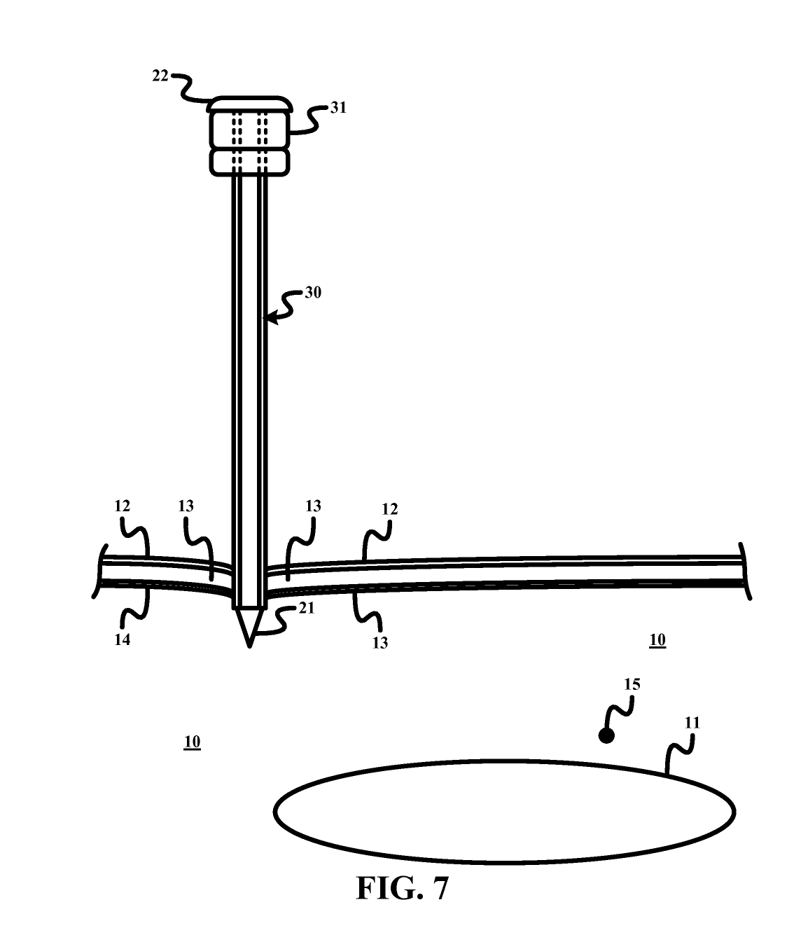

FIG. 7 illustrates an exemplary port creation phase of a collapsed lung treatment method in accordance with the present invention.

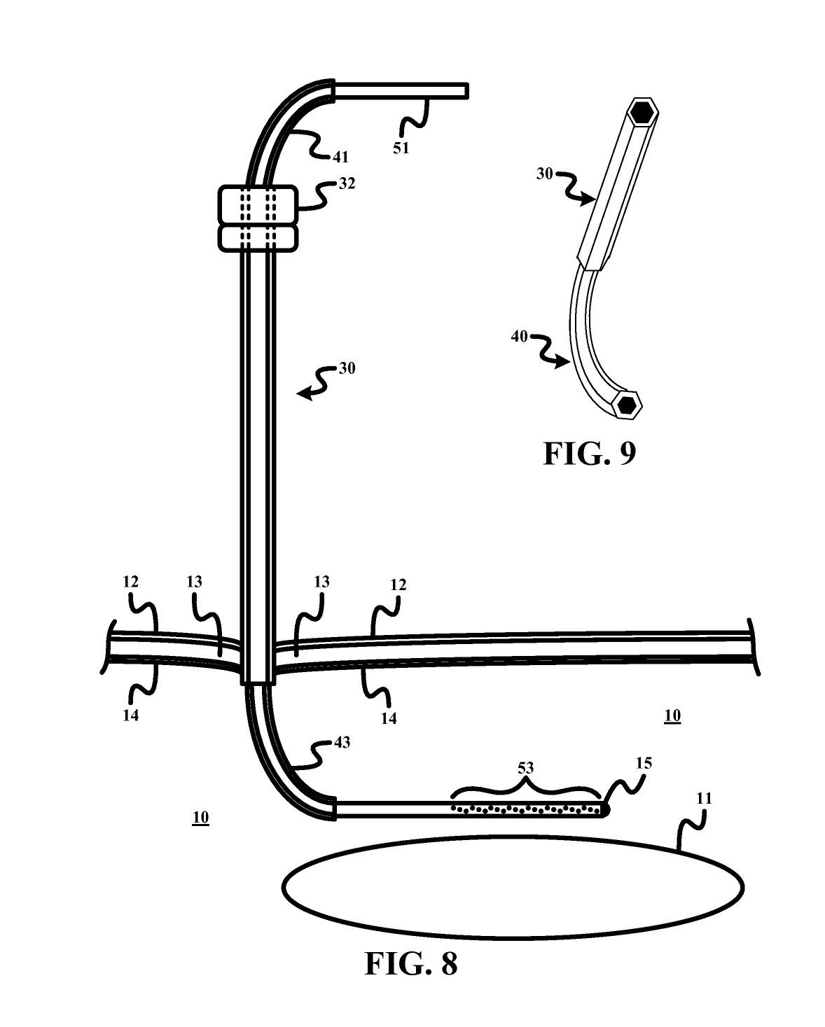

FIG. 8 illustrates an exemplary air suction phase of a collapsed lung treatment method in accordance with the present invention.

FIG. 9 illustrates an exemplary interlocking of a pleural port tube shown in FIG. 3 and the target orientation tube shown in FIG. 4.

The present invention is premised on a standardized set of cannula tubes having a fixed orientation as the cannula tube(s) are extended in a direction of a target location within a pleural cavity of a patient. The fixed orientation is particularly important in the context of a cannula tube having a longitudinal non-zero curvature (e.g., an arc) along a portion or an entirety of the cannula tube. To facilitate an understanding of the present invention, an exemplary surgical tool set (FIGS. 2-6) for treating a collapsed lung condition will be described herein in the context of a port creation phase (FIG. 7) and a fluid suction phase (FIG. 8) of a collapsed lung treatment method of the present invention.

Specifically, the surgical tool set employs a trocar 20 (FIG. 2) and a nested cannula including a pleural port tube 30 (FIG. 3), a target orientation tube 40 (FIG. 4) and a fluid suction tube 50 (FIG. 5). The tubes are fabricated from a material exhibiting desirable levels of flexibility/elasticity. For example, the material may be Nitinol, which has superelastic properties that allow the Nitinol to bend when a force is applied and to return to its original shape once the force is removed. Alternatively, the tubes may be fabricated from a polymer, such as, for example, polycarbonate, Delrin or Hostaform.

Referring to FIGS. 2, 3 and 7, during the port creation phase of the collapsed lung treatment method, trocar 20 and pleural port tube 30 are configured and dimensioned to facilitate a nesting of a trocar 20 within pleural port tube 30 whereby a tip 23 extends out of a distal end of pleural port tube upon an abutting of a cap 22 of trocar 22 against a handle 31 of pleural port tube 30. A nesting of trocar within pleural port tube 30 enables tip 21 to be utilized to puncture a port through skin 12, muscle layer 13 and chest wall 14 into pleural cavity 10 of the patient as shown in FIG. 7. Pleural port tube 30 is secured within the port upon a removal of trocar 20 from being nested within pleural port tube 30 to facilitate target orientation tube 40 (FIG. 4) and fluid suction tube 50 (FIG. 5) to reach a target location 15 adjacent to the collapsed lung 11.

In practice, trocar 20 and pleural port tube 30 may be configured and dimensioned to interlock during a nesting of a trocar 20 within pleural port tube 30 to impede or eliminate any rotation of trocar 20 within pleural port tube 30. For example, in the illustrated embodiments, trocar 20 and pleural port tube 30 have polygonal cross-sectional shapes that are dimensioned to interlock trocar 30 with pleural port tube 30 as trocar 20 is being nested within pleural port tube 30. Additional interlocking configurations of trocar 20 and pleural port tube 30 include a keyed configuration (meaning there are stops prohibiting over extending the tube?) or a rigid configuration.

Also in practice, pleural port tube 30 may be a longitudinally straight tube as shown in FIG. 3 or alternatively a longitudinally curved tube along the entirety of pleural port tube 30 or preferably curved only at the distal end of pleural port tube 30, forming a ‘J’ shape.

Referring to FIGS. 3-6 and 8, during the port creation phase of the collapsed lung treatment method, target orientation tube 40 is advanced within pleural port tube 30 in a direction of target location 15 whereby a distal end 43 of target orientation tube 40 extends from pleural port tube 30. Pleural port tube 30 and target orientation tube 40 are configured and dimensioned to interlock to achieve a fixed orientation of target orientation tube 40 relative to pleural port tube 30 as shown in FIGS. 8 and 9. For example, in the illustrated embodiments, pleural port tube 30 and target orientation tube 40 have polygonal cross-sectional shapes that are dimensioned to target orientation tube 40 with pleural port tube 30 as target orientation tube 40 is being advanced through pleural port tube 30.

In practice, target orientation tube 40 may be a longitudinally curved tube that is shown in FIG. 4 in a nesting state within pleural port tube 30 with a proximal section 41 and a distal section 43 remaining longitudinal curved sections while nested section 42 is straightened by pleural port tube 30. Alternatively, only distal section 43 of target orientation tube 40 may be longitudinally curved section, forming a ‘J’ shape.

Subsequent to the advancement of target orientation tube 40 within pleural port tube 30, fluid suction tube 50 is advanced within target orientation tube 40 in a direction of target location 15 whereby a distal end 53 of fluid suction tube 50 extends from target orientation tube 40 and a plurality of perforated holes of distal end 53 as best shown in FIG. 6 facilitate a suctioning of air from within pleural cavity 10.

In practice, fluid suction tube 50 may be a longitudinally straight tube as shown in FIG. 5 with an intermediate section 51 that longitudinally curves adjacent a proximal section 51 and a distal section 53 due to the longitudinally curvature of proximal section 41 and distal section 43 of target orientation tube 40. Alternatively, distal section 53 of air section tube 50 may be longitudinally curved section, forming a ‘J’ shape.

In an alternative embodiment of fluid suction tube 50, distal end section 53 may be integrated with distal section 43 of target orientation tube 40 to create a fluid suction tube having a perforated air suction section 53 and a target orientation section 40.

In practice, intermediate section 52 of fluid suction tube may include perforated hole(s) whereby a gap between target orientation tube 40 and fluid suction tube 50 may be utilized to suction air from pleural cavity 10, particularly if an inflating lung 11 starts to cover the perforated hole(s) of distal section 53 and bend fluid suction tube 50. Furthermore, a tip of distal section 53 is preferably rounded or covered by a soft material (e.g., rubber).

Also in practice, a standard atlas of a respiratory region of a body, human or animal, may be utilized to identify target location 15 spaced from collapsed lung 11 to prevent any potential puncturing of collapsed lung 11, and pleural port tube 30, target orientation tube 40 and fluid suction tube 50 may be configured and dimensioned with the objective of getting as close as possible to, if not reaching, target location 15. As such, the nested cannula may be commercially provided in different size sets to cover a dimensional range of respiratory regions from small (e.g., for a baby or a toddler) to medium (e.g., for teenagers and your adults to large (e.g., for adults).

Furthermore in practice, if practical in view of the collapsed lung condition of the patient, an imaging of the respiratory region may be utilized to track the translation of target orientation tube 40 and fluid suction tube 50 in the direction of the target location and to track a withdrawal of target orientation tube 40 and fluid suction tube 50 as lung 11 is inflated. Alternatively, a user of the surgical tool set may use experience and skill in tracking a translation of target orientation tube 40 and fluid suction tube 50 in the direction of the target location and a withdrawal of target orientation tube 40 and fluid suction tube 50 as lung 11 is inflated.

Please note the terms “pleural port”, “target orientation” and “fluid suction” are means for differentiating the various tubes of the nested cannula and are not intended to limit the scope of the nested cannula in accordance with the present invention.

Referring to FIGS. 2-9, those having ordinary skill in the art will appreciate numerous benefits of the present invention including, but not limited to, a rapid correction of a collapsed lung condition (e.g., pneumothorax treatment) by providing a standardized nested cannula design that may be used immediately upon diagnosis of the collapsed lung condition.

While various embodiments of the present invention have been illustrated and described, it will be understood by those skilled in the art that the embodiments of the present invention as described herein are illustrative, and various changes and modifications may be made and equivalents may be substituted for elements thereof without departing from the true scope of the present invention. In addition, many modifications may be made to adapt the teachings of the present invention without departing from its central scope. Therefore, it is intended that the present invention not be limited to the particular embodiments disclosed as the best mode contemplated for carrying out the present invention, but that the present invention includes all embodiments falling within the scope of the appended claims.

1. A surgical tool set for treating a collapsed lung condition, the surgical tool set comprising:
a trocar configured to puncture a port into a pleural cavity of a patient; and
a nested cannula including a pleural port tube and a fluid suction tube to reach a target location within the pleural cavity of the patient,
wherein the trocar and the pleural port tube are cooperatively configured and dimensioned to secure the pleural port tube within the port into the pleural cavity of the patient in response to the trocar being nested within the pleural port tube as the trocar is utilized to puncture the port into the pleural cavity of the patient,
wherein the pleural port tube and the fluid suction tube are cooperatively configured and dimensioned to fix an orientation of the fluid suction tube relative to the pleural port tube as the fluid suction tube advanced through the pleural port tube in a direction of the target location within the pleural cavity of the patient, and
wherein the fluid suction tube includes at least one perforated hole to facilitate a suctioning of fluid relative to the target location within the pleural cavity of the patient into the fluid suction tube.

2. The surgical tool set of claim 1, wherein the pleural port tube-and the fluid suction tube interlock to fix the orientation of the fluid suction tube relative to the pleural port tube as the fluid suction tube is advanced through the pleural port tube in the direction of the target location within the pleural cavity of the patient.

3. The surgical tool set of claim 1, wherein the fluid suction tube includes:
an target orientation section configured to interlock with the pleural port tube to fix the orientation of the fluid suction tube relative to the pleural port tube as the fluid suction tube is advanced through the pleural port tube in the direction of the target location within the pleural cavity of the patient, and
a fluid suction section including the at least one perforated hole.

4. The surgical tool set of claim 3, wherein the target orientation section is longitudinally curved.

5. The surgical tool set of claim 3, wherein the air suction section longitudinally straight.

6. The surgical tool set of claim 1, wherein the trocar and the pleural port tube interlock in response to the trocar being nested within the pleural port tube.

7. The surgical tool set of claim 1, wherein the pleural port tube longitudinally straight.

8. A surgical tool set for treating a collapsed lung condition, the surgical tool set comprising:
a trocar configured to puncture a port into a pleural cavity of a patient; and
a nested cannula including a pleural port tube, a target orientation tube and a fluid suction tube to reach a target location within the pleural cavity of the patient,
wherein the trocar and the pleural port tube are cooperatively configured and dimensioned to secure the pleural port tube within the port into the pleural cavity of the patient in response to the trocar being nested within the pleural port tube as the trocar is utilized to puncture the port into the pleural cavity of the patient,
wherein the pleural port tube and the target orientation tube are cooperatively configured and dimensioned to fix an orientation of the target orientation tube relative to the pleural port tube as the target orientation tube is advanced through the pleural port tube in a direction of the target location within the pleural cavity of the patient,
wherein the target orientation tube and the fluid suction tube are cooperatively and dimensioned to translate the fluid suction tube through a fixedly oriented target orientation tube in the direction of the target location within the pleural cavity of the patient, and
wherein the fluid suction tube includes at least one perforated hole to facilitate a suctioning of fluid relative to the target location within the pleural cavity of the patient into the fluid suction tube.

9. The surgical tool set of claim 8, wherein the pleural port tube and the target orientation tube interlock to fix the orientation of the target orientation tube relative to the pleural port tube as the target orientation tube is advanced through the pleural port tube in the direction of the target location within the pleural cavity of the patient.

10. The surgical tool set of claim 9, wherein the target orientation tube is longitudinally curved.

11. The surgical tool set of claim 8, wherein the target orientation tube and the fluid suction tube interlock to fix the orientation of the fluid suction tube relative to the pleural port tube as the fluid suction tube is advanced through the target orientation tube in the direction of the target location within the pleural cavity of the patient.

12. The surgical tool set of claim 11, wherein the target orientation tube is longitudinally curved.

13. The surgical tool set of claim 11, wherein the fluid suction tube is longitudinally straight.

14. The surgical tool set of claim 8, wherein the trocar and the pleural port tube interlock in response to the trocar being nested within the pleural port tube.

15. The surgical tool set of claim 8, wherein the pleural port tube is longitudinally straight.

16. (canceled)

17. (canceled)

18. (canceled)

19. (canceled)

20. (canceled)
